(19) United States
(12) Patent Application Publication
(10) Pub. No.: US 2022/0170119 A1
(43) Pub. Date: Jun. 2, 2022

(54) Signaling Pathway of Cannabidiol (CBD) for Prevention and Treatment of COVID-19

(71) Applicant: Hemix Biotech Limited, Hong Kong (HK)

(72) Inventors: Zhongjun Zhou, Hong Kong (HK); Sheung Kin Ken Wong, Hong Kong (HK); Hin Chu, Hong Kong (HK); Xuelai Wang, Hong Kong (HK); Jie Zhou, Hong Kong (HK); Cun Li, Hong Kong (HK); Leung Kris Ying, Hong Kong (HK); Jian Zhang, Shanghai (CN)

(21) Appl. No.: 17/540,259

(22) Filed: Dec. 2, 2021

Related U.S. Application Data
(60) Provisional application No. 63/120,359, filed on Dec. 2, 2020.

(51) Int. Cl. C12Q 1/70; A61K 31/05; A61P 31/14; C12Q 1/686; C12Q 1/6888

(52) U.S. Cl. CPC C12Q 1/70; A61K 31/05; C12Q 2600/158; C12Q 1/686; C12Q 1/6888; A61P 31/14

(57) ABSTRACT

The invention relates to the technical field of medicine, and provides a method of screening and identifying small molecule with antiviral properties. The invention has screened and identified Cannabidiol (CBD), which can be used for prevention of viral infections. The invention also examined and verified that CBD can be used for prevention of COVID-19 disease, by a key signaling pathway of upregulating the expression of the TRIB3 gene. The invention also determined that the optimal dosage of CBD treatment for prevention of COVID-19 disease is 5 μM-10 μM.

### CROSS-REFERENCE TO RELATED APPLICATIONS

This application claims priority to U.S. Provisional Patent Application No. 63/120,359, entitled “Cannabidiol (CBD) as Chemical for Preventing the Infection of SARS-CoV-2 and Treating COVID-19”, filed on Dec. 2, 2020, the disclosure of which is incorporated by reference in its entirety.

### BACKGROUND OF THE INVENTION

The present invention relates to the technical field of medicine. More particularly, the present invention is in the technical field of screening and identifying small molecules that have antiviral properties.

By the middle of 2021, over 255 million people worldwide have been infected by SARS-CoV-2 (also known as the Coronavirus), the virus that causes COVID-19 diseases, and more than 5 million deaths from the coronavirus, according to the data from government health ministries and other authoritative sources. The newly emerged variants, e.g., B.1.617 variant and B.1.1.7 variant, increased the transmission of the diseases and contributed to the resurgent of the viral infection in countries such as India, Japan, UK, Peru, Brazil.

Though vaccines for Covid-19 disease were developed and proved for emergency use in many countries, issues such as safety and sufficient efficacy, long-term immunity, and supply chains hamper the effort for the massive vaccination, especially in low-income countries. Evidence for effective drugs is also limited. It was therefore desirable to find a rapid screening and identifying method which could be used to find more small molecules with antiviral properties that could be used to prepare drugs for the prevention of coronavirus infections.

UK RECOVERY trial and World Health Organization solidarity trial showed that low dose dexamethasone reduces deaths in patients hospitalized with severe Covid-19 diseases. This is by far the only drug that showed efficacy in preventing deaths in COVID-19 patients which was recommended by WHO. Remdesvir might reduce the length of hospitalization but not death, though FDA issued emergency use for Remdesvir to treat hospitalized COVID-19 patients. Authorities in China, Japan, South Korea, Hong Kong, and European Union also recommended Remdesivir in treating Covid-19 patients. Efficacy in treating COVID-19 by other existing drugs such as Lopinavir-ritonavir, Favipiravir, Ivermectin is not conclusive.

Analysis and statistics of COVID-19 infection cases shown that the elderly are more susceptible to COVID-19 infection, and the death rate for the elderly is higher than the other groups of age. COVID-19 requires the host's cellular machinery to infect and replicate. By analyzing Genotype-Tissue Expression (GTEx) data, a bioinformatic study was carried out to identify aging-associated genes that have potential interaction with the coronaviruses. The gene Tribbles homolog 3 (TRIB3) was found to be decreased during aging in males. Using publicly available lung single-cell data, it was found that TRIB3 has high expression in alveolar epithelial cells. TRIB3 was reported to decrease viral entry and replication of the Hepatitis C virus. Therefore, loss of TRIB3 in the elderly may contribute to their high infection rate of COVID-19.

At present the main methods of preventing infection with novel coronavirus include Vaccination, social distancing, washing hands frequently and wearing masks in public. How to quickly screen and identify effective drugs with antiviral activity is a challenge faced by the researcher.

### SUMMARY OF THE INVENTION

In recent years, Cannabidiol (CBD), a major non-psychotropic phytocannabinoid found in cannabis, has been used in treatments and clinical trials in various diseases including chronic pain, anorexia, nausea, spasticity and multiple sclerosis. Recently, CBD was suggested to be a potential treatment for COVID-19 due to its anti-inflammatory effects. However, the detailed key signaling pathway is unknown.

According to one aspect of the invention, a key signaling pathways by which CBD could prevent virus infection is provided, namely CBD could prevent infection of SARS-CoV-2, the virus causes Covid-19, by upregulating the expression of protein Tribbles Homolog 3 (TRIB3).

According to another aspect of the invention, a method for preventing COVID-19 disease caused by SARS-CoV-2 virus by using Cannabidiol to upregulating the expression of protein Tribbles Homolog 3 (TRIB3), wherein the optimal dosage of CBD is 5 μM-10 μM.

According to a further aspect of the invention, a method of screening and identifying small molecules with antiviral properties for the prevention of COVID-19 is provided.

According to a further aspect of the invention, a method of screening and identifying small molecules with antiviral properties for the prevention of COVID-19 is provided, wherein the small molecule with antiviral properties is CBD.

Unless otherwise defined, all technical and/or scientific term used herein have the same meaning as commonly understood by one of ordinary skills in the art to which the invention pertains. Although methods and materials similar or equivalent to those described herein can be used in the practice or testing of embodiments of the invention, exemplary methods and/or materials are described below. In addition, the materials, methods, and examples are illustrative only and are not intended to be necessarily limiting.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regards, the description taken with the drawings makes apparent to those skilled in the how embodiments of the invention may be practiced.

In the drawings:

FIG. 1 is a flowchart of method for screening, identifying, verifying chemicals and/or small molecules having antiviral functions and properties.

FIG. 2 illustrates the effect of CBD on proliferation of wild type and Lamin A 609 MEFs. The population doubling level (PDL) of wild type and Lamin A 609 MEFs receiving optimal dosage (10 μM) and high dosage (50 μM), which optimal dosage significantly increased PDL of both wild type and Lamin A 609 MEFs while high dosage significantly decreased PDL of both wild type and Lamin A 609 MEFs.

FIG. 3 illustrates the TRIB3 identified by RNA sequencing as one of the aging-associated virus-interacting genes which is downregulated in premature aging Lamin A mutation MEFs while upregulated by CBD treatment in wild type and Lamin A 609 MEFs. Graph showing fragments per Kilobase of transcript per million mapped reads (fpkm) of TRIB3 in wild type and Lamin A 609 MEFs receiving CBD treatment.

FIG. 4 illustrates the results of qPCR analysis of TRIB3 in lung tissue of Lamin A 609 mice after receiving 2-month CBD treatment compared to wild type and Lamin A 609 control mice.

FIG. 5 illustrates the results of qPCR analysis of viral gene copy number in cell lysate of Huh7 liver cells upon CBD treatment.

FIG. 6 illustrates the results of qPCR analysis of viral gene copy number in supernatant of Huh7 liver cells upon CBD treatment.

FIG. 7 illustrates the results of luciferase assay analysis of SARS-Cov-2 pseudovirus infection of Caco2 cells upon CBD treatment.

FIG. 8 illustrates the proliferation rate of Caco2 cells with CBD treatment.

FIG. 9 illustrates that the 5 μM CBD treatment did not affect expression of KI67.

FIG. 10 illustrates the results of CBD upregulated TRIB3 and ATFS in Caco2 cells.

FIG. 11 illustrates the results of 5 μM CBD treatment upregulated TRIB3 and ATFS.

FIG. 12 illustrates the results of qPCR analysis of viral gene copy in nasal turbinate of SARS-CoV-2 infected golden hamster upon CBD treatment.

FIG. 13 illustrates the results of qPCR analysis of viral gene copy in lung of SARS-CoV-2 infected golden hamster upon CBD treatment.

FIG. 14 illustrates the results of Masson Trichrome staining of Covid-19 infected golden hamster lung treated with control or CBD.

### LIST OF ABBREVIATIONS

| | |
|---|---|
| CBD | Cannabidiol |
| SARS-CoV-2 | Severe acute respiratory syndrome coronavirus 2 |
| COVID-19 | Coronavirus disease 2019 |
| WHO | World Health Organization |
| FDA | U.S. Food and Drug Administration |
| GTEx | Genotype-tissue expression |
| TRIB3 | Tribbles homolog 3 |
| MEFs | Mouse embryonic fibroblasts |
| API | Active pharmaceutical ingredient |
| PDL | Population doubling level |
| DMEM | Dulbecco's modified Eagle's medium |
| FBS | Fetal bovine serum |
| HGPS | Hutchinson Gilford progeria syndrome |
| PBS | Phosphate buffered saline |
| DEPC | Diethylpyrocarbonate |
| qPCR | Quantitative polymerase chain reaction |
| IP | Intraperitoneal injection |
| PEG | Polyethylene glycol |
| DMSO | Dimethyl sulfoxide |
| nCoV | Novel coronavirus |
| WT | Wild type |
| MUT | Lamin A G609G mutation |
| Huh-7 | Human hepatoma-derived Huh-7 cell |
| Caco-2 | Immortalized cell line of human |
| | colorectal adenocarcinoma cells |
| KI67 | Ki-67 (protein) |
| DAPI | 4′, 6-diamidino-2-phenylindole |
| ATF5 | Activating Transcription Factor 5 |
| MOI | Multiplicity of infection |
| HEPES | 4-(2-hydroxyethyl)-1-piperazineethanesulfonic acid |

### DETAILED DESCRIPTION OF THE INVENTION

The present invention will be further described below in connection with following embodiments. It should be understood that the following embodiments are intended to illustrate the invention only, but are not intended to limit the scope of protection of the present invention. Where specific conditions are not indicated in the following embodiments, they are performed according to conventional conditions or with reference to the manufacturer's protocols. The instruments or reagents used, where the manufacturer is not specified, are conventional products available commercially.

According to a first aspect of the present invention, a method of using Cannabidiol to upregulate the expression of the TRIB3 gene and thereby prevent viral infection is provided. TRIB3 (Tribbles Homolog 3) is identified as one of the aging-associated virus-interacting genes which is downregulated in premature aging Lamin A mutation MEFs. In order to verify that Cannabidiol upregulates TRIB3 gene expression and thus prevents viral infection, the following methods and materials are used:

CBD was dissolved in 5% DMSO and 5% polyethylene glycol (PEG) in sterile saline and stored at −80° C. for up to 1 month. 2 months old LMNA^{G609G/G609G} mice were selected used. The mice then received an intraperitoneal injection (IP) twice a week until they reached the humane point.

Dissecting mice for preparation of tissues extraction of RNA, the mice were sacrificed by cervical dislocation under anesthesia by intraperitoneal (IP) injection of 100 mg/kg ketamine and 16 mg/kg xylazine in sterile water. Major organs, including skin, muscle, intestine, kidney, spleen, liver, lung, heart, aorta, were collected. Half of the tissues were snap-frozen in liquid nitrogen and stored at −80° C. for extraction of RNA.

Carrying out RNA extraction, DNase digestion, reverse transcription and qPCR analysis according to the following steps. For tissues, 50 mg of tissues were added to 1 mL TRIzol and homogenized using tissue homogenizer on ice. After incubation at room temperature for 10 minutes, the samples were centrifuged at 4° C. at 12000 rcf for 10 minutes. The supernatants were collected to Eppendorf tubes. 200 μl of chloroform was added to each sample, and the tubes were shaken vigorously by hand for 15 seconds. The samples were incubated at room temperature for 5 minutes and then centrifuged at 4° C. at 12000 rcf for 15 minutes. After centrifugation, the solution was separated into 3 layers. 450 μl of the clear top layer was collected, and 500 μl of isopropanol was added. After incubation at room temperature for 10 minutes, the samples were centrifugated at 4° C. at 12000 rcf for 10 minutes. RNA pellets would be visible at the bottom of the tubes. The RNA pellets were washed by 75% ethanol in diethylpyrocarbonate (DEPC) water. The RNA in 75% ethanol can be stored at −20° C. for 1 year.

For RNA using for quantitative polymerase chain reaction (qPCR), the samples were centrifugated at 4° C. at 7500 rcf for 5 minutes. The ethanol was removed, and the samples were air-dried until the RNA pellets turned transparent. Then the RNA pellets were resuspended with DEPC water. The concentration of RNA was measured by nanodrop.

2 μg of RNA was used for DNase digestion and reverse transcription, while the rest can be stored at −80° C. for one month. For DNase digestion, 2 μg of RNA was used for each reaction using Promega® RQ1 Rnase-Free Dnase following the manufacturer's protocol.

Carrying out DNase digestion to remove the DNA contaminant in the RNA samples, which prevents the amplification of genomic DNA in qPCR. The DNase digested RNA samples were then used for reverse transcription using Thermo Scientific® High Capacity cDNA Reverse Transcription Kit with RNase Inhibitor following manufacturer's protocol.

The transcribed cDNA from the previous step was diluted 4 times with Milli-Q® water. Then the samples were ready for qPCR. The unused samples were stored at −20° C. For qPCR test, 0.5-1 μl of cDNA was used for each 10 μl reaction. The Delta-Delta-Ct (also known as 2^{-ΔΔCt}) value was calculated by the qPCR machine based on the signal of target genes normalized with housekeeping genes.

RNA sequencing is carried out wherein the RNA samples were extracted using TRIzol and kept in 75% ethanol in diethylpyrocarbonate (DEPC) water at −20° C. For each condition, two biological replicates were prepared for RNA sequencing. The RNA samples were dissolved in DEPC water and carried out quality control analysis. The RNA quantitation was done using Nanodrop™ and Agilent® 2100 Bioanalyzer. The RNA integrity was measured using Agilent® 2100 Bioanalyzer. The RNA purity was measured using agarose gel electrophoresis and Agilent® 2100 Bioanalyzer.

The samples that had concentration over 50 ng/μl for volume over 20 μl, RIN value over 6.3, and OD260/280 value over 2.0 were considered to pass the quality control analysis and were used for RNA sequencing. Illumina sequencing system was used for paired-end reads of read lengths PE150. 6 GB of raw data was acquired from each sample. Bioinformatic analysis was performed which included data quality control, alignment, gene expression level analysis, differential gene expression analysis, and functional analysis.

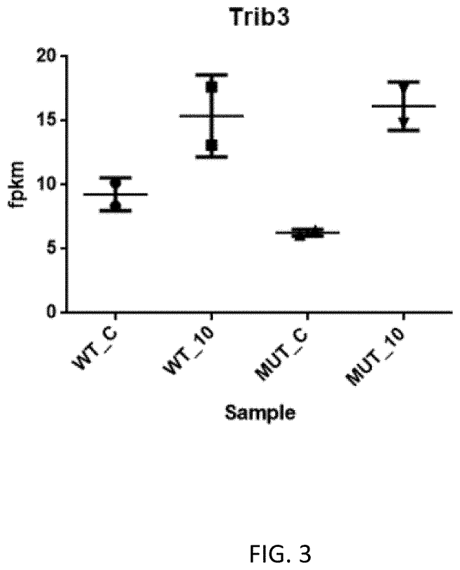

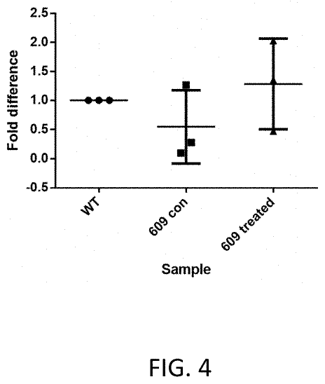

Referring to FIG. 3 and FIG. 4, the RNA sequencing results showed that CBD treatment in premature aging Lamin A 609 mouse embryonic fibroblasts (MEFs) can increase the expression levels of TRIB3 which its expression is downregulated in premature aging Lamin A 609 MEFs.

Upon the above steps were done, the method and analysis results proved that CBD treatment can upregulate the expression of TRIB3 gene, since TRIB3 can interact with the virus, therefore CBD treatment is helpful for preventing viral infection. In order to verify the inhibitory effect of CBD on nCoV virus infection, the following specific steps and materials were used in the present invention:

To examine CBD inhibitory effects on nCoV infection, Huh7 cells were chosen. 2.0×10⁵ cells/well of Huh7 cells were seeded to 24-well plate. The Huh7 cells were treated with DMSO as control, 2.5 μM, 5 μM or 10 μM CBD for 1 hour. The Huh7 cells were then infected with 0.01 MOI of nCoV virus for 1 hour. The inoculum was discarded and the plate was washed with 1 mL PBS twice. The Huh7 cells were then cultured in DMEM/F-12 and treated with DMSO as control, 2.5 μM, 5 μM or 10 μM CBD. After 2, 24 and 48 hours after infection, the supernatant and cell lysate were collected for qPCR analysis for determination of viral gene copy number.

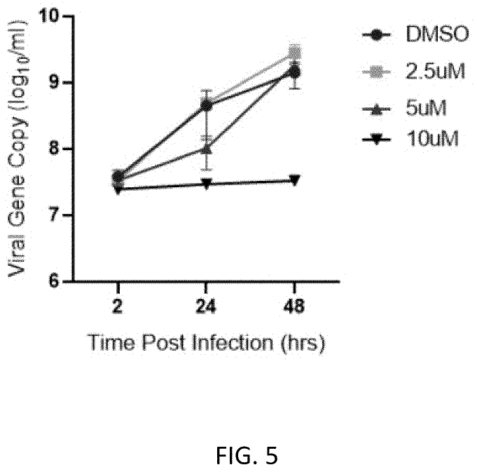

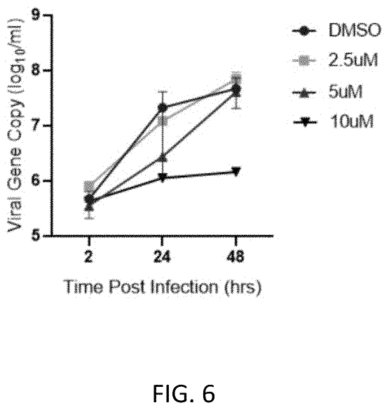

Referring to FIGS. 5 and 6, the present invention employed qPCR analysis to detect the number of viral gene copies in the cell lysate and supernatant of Huh7 hepatocytes treated with CBD. The results demonstrated that CBD inhibited nCoV virus infection and the optimal dosage of CBD is 5 μM.

To further verify that CBD treatment has inhibitory effect on nCoV virus infection through upregulating TRIB3 gene expression, the present invention used further other different cells and methods through embodiments, as illustrated by the following specific steps and materials with reference to the accompanying figures.

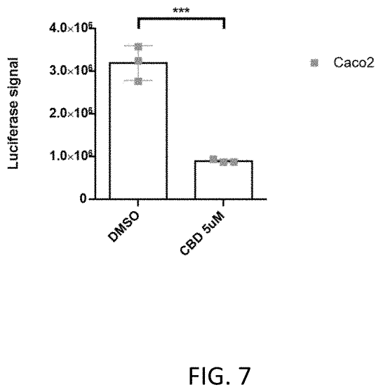

For example, in order to examine the CBD inhibitory effects on nCoV infection in other organs, types of cells, the similar method was used in intestine cells (Caco-2) and golden hamster. Referring to FIG. 7, we have studied the inhibitory effects on SARS-Cov-2 pseudovirus infection of Caco2 cells, by the luciferase assay analysis. The results shown that CBD treatment can significantly reduce the SARS-Cov-2 pseudovirus infection by 72%. This proved that CBD could suppress SARS-Cov-2 infection which could therefore be used for prevention of COVID-19.

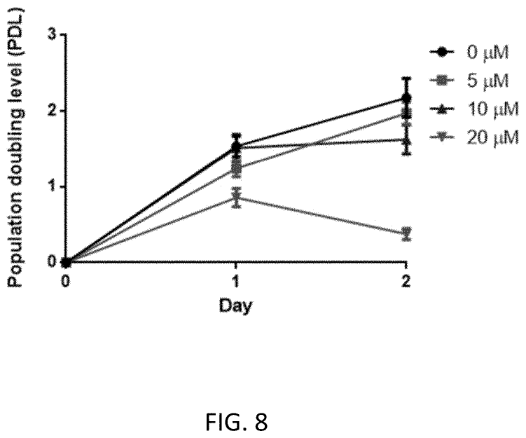

Referring to FIG. 8, which shown the proliferation rate of Caco-2 cells with CBD treatment. We have compared the population doubling level (PDL) of Caco-2 cells with CBD treatment at four different concentration, namely, 0 μM CBD, 5 μM CBD, 10 μM CBD, and 20 μM CBD. The results shown that 5 μM CBD treatment didn't affect proliferation of Caco-2 cells shown by population doubling level (PDL). This indicated that at the concentration of 5 μM, CBD would not cause adverse effect on cells that are not infected by COVID-19. Therefore, the COVID-19 virus suppression effect observed in FIG. 7 was not caused by toxicity of CBD.

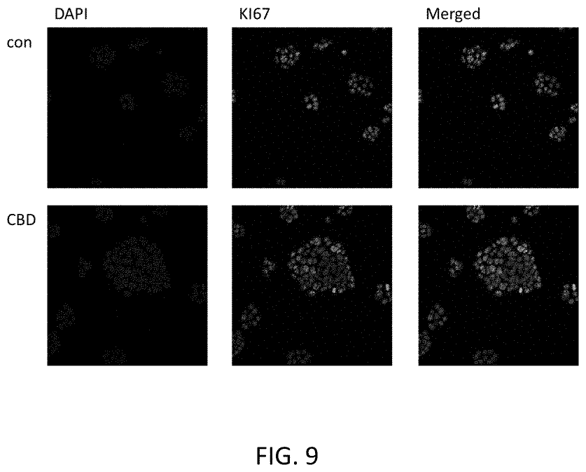

Referring to FIG. 9, to confirm that the COVID-19 virus suppression effect observed in FIG. 7 was not caused by toxicity of CBD, we have analyzed the expression of KI67 with CBD treatment at the concentration of 5 μM CBD. The results shown that 5 μM CBD treatment didn't affect proliferation of Caco2 cells shown by KI67. Similar to FIG. 7, the results indicated that at the concentration of 5 μM, CBD would not cause adverse effect to cells that are not infected by COVID-19. It is confirmed that the COVID-19 virus suppression effect observed in FIG. 7 was not caused by toxicity of CBD.

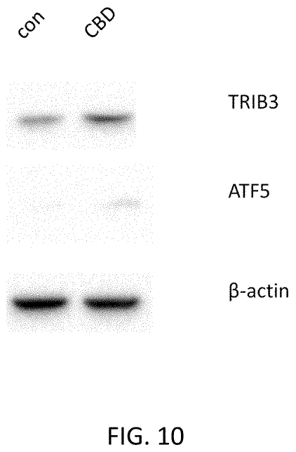

Referring to FIG. 10, we have further analyzed the mechanism of CBD treatment on Caco-2 cells by comparing the expression level of TRIB3 and AFT5 in Caco-2 cells by the CBD treatment. The results shown that CBD treatment can significantly upregulate TRIB3 and ATF5 in Caco2 cells, which contribute to CBD's anti-COVID-19 effects. TRIB3 has important functions in preventing virus infection and the RNA sequencing results also showed TRIB 3 was downregulated in premature aging cells. Similarly, ATF5 was also identified by the RNA sequencing results and it is closely associated with TRIB3. The results as shown in FIG. 10 proved that the expression level of TRIB3 and ATF5 upregulated by CBD treatment proved that CBD has noticeable effects on prevention of COVID-19 disease.

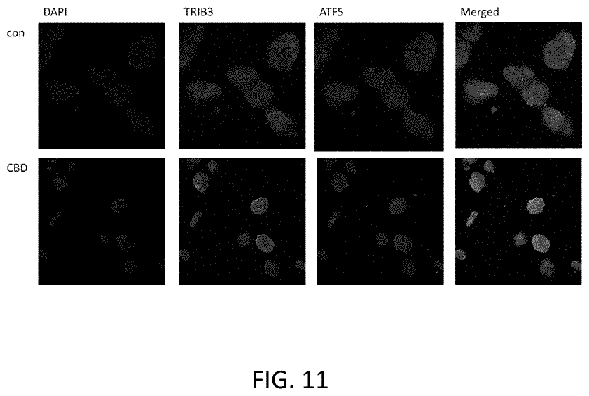

Referring to FIG. 11, we have further analyzed the mechanism of CBD treatment on Caco-2 cells by comparing the expression level of TRIB3 and AFT5 in Caco2 cells by the CBD treatment. Compared to FIG. 10, we have measured the expression of TRIB3 and AFT5 by immunofluorescence staining. The results of FIG. 11 demonstrated that CBD treatment increased TRIB3 and ATF5 levels of expression. And since the upregulation of TRIB3 and ATF5 contributes to CBD's anti-COVID-19 effects, therefore, it is proved that CBD has significant effects on prevention of COVID-19 disease.

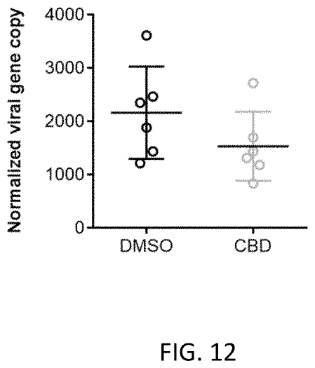

Referring to FIG. 12, we tested the CBD's anti-COVID-19 effects in nasal turbinate of golden hamster by qPCR analysis of viral gene copy in nasal turbinate of SARS-CoV-2 infected golden hamster upon CBD treatment. The results shown that CBD treatment reduced SARS-CoV-2 infection in nasal turbinate of golden hamster. This proved that CBD suppress SARS-Cov-2 infection which is helpful for prevention of COVID-19.

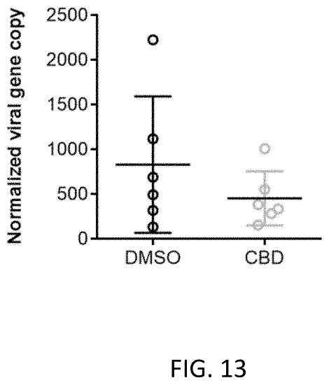

Referring to FIG. 13, we have repeated the analysis as illustrated in FIG. 12, by qPCR analysis of viral gene copy in lung of SARS-CoV-2 infected golden hamster upon CBD treatment to further confirm that CBD can suppress SARS-Cov-2 infection which is helpful for prevention of COVID-19.

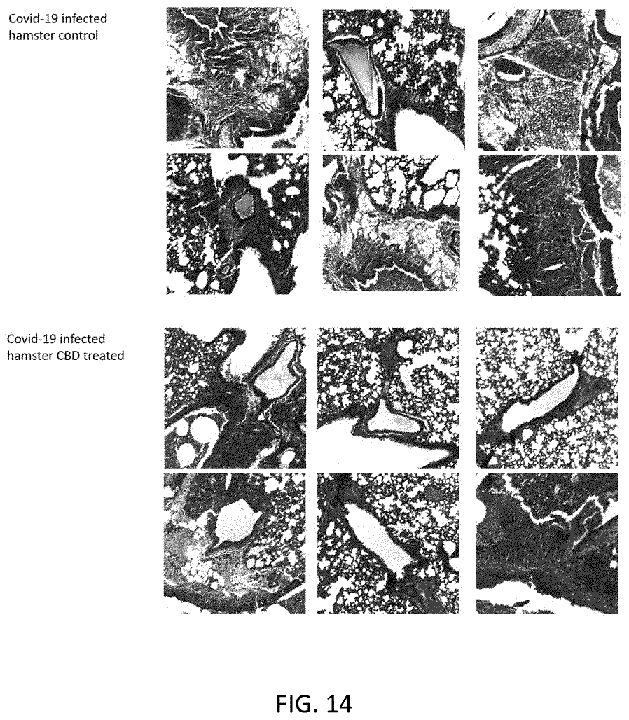

Referring to FIG. 14, we have further tested CBD's anti-COVID-19 effects by analysis of Masson Trichrome staining of Covid-19 infected golden hamster lung treated with control or CBD. As shown in FIG. 14, the blue stain in the Masson Trichrome staining implies fibrosis. In the lung of Covid-19 infected golden hamster treated with CBD, area and intensity of blue stain were significantly lowered compared to that of control. It is indicated that that CBD prevents lung fibrosis after Covid-19 infection which is helpful to accelerate the recovery process of Covid-19 patients.

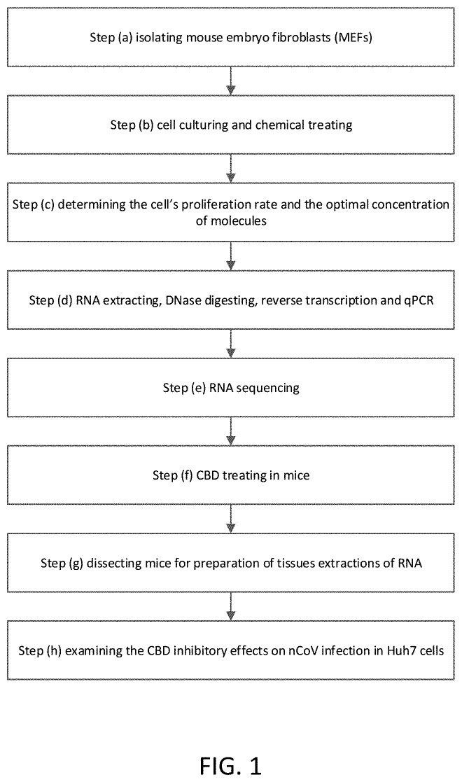

Through the above methods and materials of different cells and different CBD dosage, the present invention also provides a method for screening and identifying small molecules with antiviral properties. FIG. 1 is a flow diagram of a method of screening and identifying small molecules with antiviral properties. Referring to FIG. 1, the method comprised of the steps of (a) isolating mouse embryo fibroblasts (MEFs), (b) cell culturing and chemical treating, (c) determining the cell's proliferation rate and the optimal concentration of molecules, (d) RNA extracting, DNase digesting, reverse transcription and qPCR, (e) RNA sequencing, (f) CBD treating in mice, (g) dissecting mice for preparation of tissues extractions of RNA, and (h) examining the CBD inhibitory effects on nCoV infection in Huh7 cells.

Using the method as described in FIG. 1, whether a small molecule has antiviral function or properties can be quickly screened and identified. According to one embodiment of the present invention, CBD was chosen to verify the effects on prevention of nCoV infection. The effects have been verified based on Huh7 cells.

Materials and methods of the embodiment is described as below in detail:

### Step (a)—Isolation of Mouse Embryo Fibroblasts (MEFs)

In the step of isolating mouse embryo fibroblasts, the Heterozygous (LMNA^{G609G/+}) mice were set for mating. The date of pregnancy of mice was identified by checking for vaginal plug in the morning following the day of mating. When the vaginal plug was found, the mouse was considered to be pregnant for 0.5 days (E0.5). When the embryos reached E12.5 to E13.5, the pregnant mice were sacrificed, and the embryos were isolated from the mice inside tissue culture hood using sterile utensils.

The embryos were placed in phosphate buffered saline (PBS). Their head and liver were removed. A portion of the head was used for genotyping to identify the genotype of each embryo. The body of each embryo was transferred to 1 mL of 0.1% Trypsin-EDTA solution in a well of 12-well plate. Using sterile scissors, the embryos were cut into small pieces.

The 12-well plate was then placed in a 37° C. incubator for 10 minutes. Followed by vigorous pipetting of the embryos in Trypsin-EDTA solution until the embryos wholly dissolved into the solution. The 12-well plate was then placed in 37° C. incubator for 5 minutes. The homogenized solution was then transferred to 9 mL of Gibco's High Glucose Dulbecco's Modified Eagle's Medium (DMEM) supplemented with sodium bicarbonate (3.7 g/L), HEPES (6 g/L), 10% fetal bovine serum (FBS) and penicillin-streptomycin (100 units/mL). The isolated MEFs were considered to be at passage 0 (P0). P3 MEFs were used in the invention.

### Step (b)—Cell Culture and Chemical Treatment

In the step of cell culturing and chemical treating, the primary mouse embryonic fibroblasts (MEFs) were cultured in Gibco's High Glucose Dulbecco's Modified Eagle's Medium (DMEM) supplemented with sodium bicarbonate (3.7 g/L), and 10% fetal bovine serum (FBS). Huh7 cells were cultured in Dulbecco's Modified Eagle Medium/Nutrient Mixture F-12 (DMEM/F-12) supplemented with sodium bicarbonate (2.438 g/L), and 10% fetal bovine serum (FBS).

### Step (c)—Determination of Cell's Proliferation Rate and the Optimal Concentration of Molecules

In the step of determining cells' proliferation rate and the optimal concentration of molecules, the primary mouse embryonic fibroblasts (MEFs) at passage 3 were counted by using LUNA-II Automated Cell Counter. 1.0×10⁵ cells were seeded to a well of 6-well plate. The cells were treated with DMSO as control, 10 μM or 50 μM CBD.

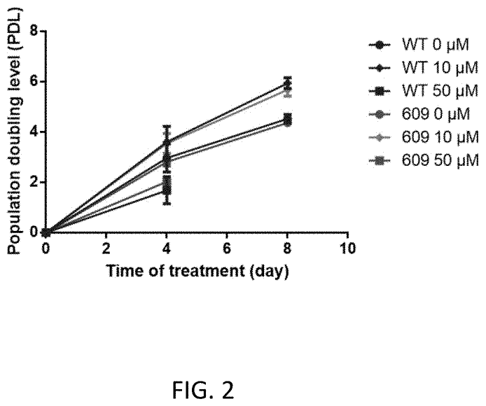

Referring to FIG. 2, after 4-day and 8-day treatment, the cells were counted again using automated cell counter. The population doubling level (PDL), which reflects the proliferation rate of cells, was calculated by the formula: n=3.32 (log UCY−log I)+X, where n=the PDL number, UCY=the cell yield at that time point, I=the initial cell number, and X=the doubling level of the cells used to initiate the subculture being quantitated.

The population doubling level (PDL) of wild type and Lamin A 609 MEFs receiving optimal dosage (10 μM) and high dosage (50 μM) was plotted, which optimal dosage significantly increased PDL of both wild type and Lamin A 609 MEFs while high dosage significantly decreased PDL of both wild type and Lamin A 609 MEFs.

### Step (d)—RNA Extraction, DNase Digestion, Reverse Transcription, and Quantitative Polymerase Chain Reaction (qPCR)

In the step of RNA extracting, DNase digesting, reverse transcription and qPCR, for cells, the dishes were washed twice with ice-cold phosphate buffered saline (PBS), and 1 mL TRIzol was added. After incubation at room temperature for 5 minutes, the samples were collected into Eppendorf tubes.

In the step of RNA extracting, DNase digesting, reverse transcription, and qPCR, for tissues, 50 mg of tissues were added to 1 mL TRIzol and homogenized using tissue homogenizer on ice. After incubation at room temperature for 10 minutes, the samples were centrifuged at 4° C. at 12000 rcf for 10 minutes. The supernatants were collected to Eppendorf tubes. 200 μl of chloroform was added to each sample, and the tubes were shaken vigorously by hand for 15 seconds. The samples were incubated at room temperature for 5 minutes and then centrifuged at 4° C. at 12000 rcf for 15 minutes. After centrifugation, the solution was separated into 3 layers. 450 μl of the clear top layer was collected, and 500 μl of isopropanol was added. After incubation at room temperature for 10 minutes, the samples were centrifugated at 4° C. at 12000 rcf for 10 minutes. RNA pellets would be visible at the bottom of the tubes. The RNA pellets were washed by 75% ethanol in diethylpyrocarbonate (DEPC) water. The RNA in 75% ethanol can be stored at −20° C. for 1 year.

For RNA using for quantitative polymerase chain reaction (qPCR), the samples were centrifugated at 4° C. at 7500 rcf for 5 minutes. The ethanol was removed, and the samples were air-dried until the RNA pellets turned transparent. Then the RNA pellets were resuspended with DEPC water. The concentration of RNA was measured by nanodrop.

2 μg of RNA was used for DNase digestion and reverse transcription, while the rest can be stored at −80 ° C. for one month. For DNase digestion, 2 μg of RNA was used for each reaction using Promega® RQ1 Rnase-Free Dnase following the manufacturer's protocol.

DNase digestion removes the DNA contaminant in the RNA samples, which prevents the amplification of genomic DNA in qPCR. The DNase digested RNA samples were then used for reverse transcription using Thermo Scientific® High Capacity cDNA Reverse Transcription Kit with RNase Inhibitor following manufacturer's protocol.

The transcribed cDNA was diluted 4 times with Milli-Q® water. Then the samples were ready for qPCR. The unused samples were stored at −20° C. For qPCR 0.5-1 μl of cDNA was used for each 10 μl reaction. The Delta-Delta-Ct (also known as 2^{-ΔΔct}) value was calculated by the qPCR machine based on the signal of target genes normalized with housekeeping genes.

### Step (e)—RNA Sequencing

In the step of RNA sequencing, the RNA samples were extracted using TRIzol and kept in 75% ethanol in diethylpyrocarbonate (DEPC) water at −20° C. For each condition, two biological replicates were prepared for RNA sequencing. The RNA samples were dissolved in DEPC water and carried out quality control analysis. The RNA quantitation was done using Nanodrop™ and Agilent® 2100 Bioanalyzer. The RNA integrity was measured using Agilent® 2100 Bioanalyzer. The RNA purity was measured using agarose gel electrophoresis and Agilent® 2100 Bioanalyzer.

The samples that had concentration over 50 ng/μl for volume over 20 μl, RIN value over 6.3, and OD260/280 value over 2.0 were considered to pass the quality control analysis and were used for RNA sequencing. Illumina sequencing system was used for paired-end reads of read lengths PE150.

### Step (f)—CBD Treatment in Mice

In step of CBD treating in mice, 2 months old LMNA^{G609G/G609G} mice were selected and used. They received an intraperitoneal injection (IP) twice a week until they reached the humane point. Different dosages were used for the injection; 50 mg/kg of CBD was identified as the optimal dosage with the greatest extend in health span of the treated mice. CBD was dissolved in 5% DMSO and 5% polyethylene glycol (PEG) in sterile saline and stored at −80° C. for up to 1 month.

### Step (g)—Dissection of Mice for Preparation of Tissues Extraction of RNA

In the step of dissecting mice for preparation of tissues extraction of RNA, the mice were sacrificed by cervical dislocation under anesthesia by intraperitoneal (IP) injection of 100 mg/kg ketamine and 16 mg/kg xylazine in sterile water. Major organs, including skin, muscle, intestine, kidney, spleen, liver, lung, heart, aorta, were collected. Half of the tissues were snap-frozen in liquid nitrogen and stored at −80° C. for extraction of RNA.

### Step (h)—Examination of CBD Inhibitory Effects on nCoV Infection in Huh7 Cells

In the step of examining CBD inhibitory effects on nCoV infection in Huh7 cells, 2.0×10⁵ cells/well of Huh7 cells were seeded to 24-well plate. The Huh7 cells were treated with DMSO as control, 2.5 μM, 5 μM or 10 μM CBD for 1 hour. The Huh7 cells were then infected with 0.01 MOI of nCoV virus for 1 hour. The inoculum was discarded and the plate was washed with 1 mL PBS twice. The Huh7 cells were then cultured in DMEM/F-12 and treated with DMSO as control, 2.5 μM, 5 μM or 10 μM CBD. After 2, 24 and 48 hours after infection, the supernatant and cell lysate were collected for qPCR analysis for determination of viral gene copy number.

It can be seen from the above embodiments, the present invention used the method of screening and identifying small molecules with antiviral properties, have successfully screened CBD, a small molecule with antiviral properties, and verified that CBD can be used for prevention of viral infections, by upregulating the expression of the TRIB3 gene. It is also determined that the optimal dosage of CBD treatment for prevention of COVID-19 disease is 5 μM-10 μM.

Any of the features, attributes, or steps of the above described embodiments and variations can be used in combination with any of the other features, attributes, and steps of the above described embodiments and variations as desired.

Although the invention has been shown and described with respect to a certain embodiment or embodiments, it is apparent that this invention can be embodied in many different forms and that many other modifications and variations are possible without departing from the spirit and scope of this invention.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

The invention claimed is:

1. A method of identifying and verifying small molecule which having antivirus properties and being able to regulate virus-interacting genes, the method comprising:
Step (a): dissolving the small molecule in 5% DMSO and 5% PEG in sterile saline, whereby obtaining a solution of the small molecules;
Step (b): treating mice with the solution of the small molecules;
Step (c): dissecting said mice for preparation of tissues extraction of RNA;
Step (d): extracting RNA samples from the tissues extraction for qPCR testing, removing DNA contaminant in the RNA samples, using the DNase digested RNA samples for reverse transcription to obtain transcribed cDNA, and carrying out the quantitative polymerase chain reaction (qPCR) test for the transcribed cDNA;
Step (e): using RNA sequencing to detect the expression of genes to verify the small molecule which having antivirus properties and being able to regulate virus-interacting genes.

2. The method according to claim 1 wherein said small molecule in step (a) is Cannabidiol (CBD).

3. The method according to claim 2 wherein said virus-interacting gene is TRIB3 gene.

4. The method according to claim 3 wherein said Cannabidiol being able to inhibit and prevent viral infection by upregulating expression of the TRIB3 gene.

5. The method according to claim 4 wherein said viral infection is COVID-19 disease caused by nCoV virus.

6. The method according to claim 5 wherein optimal dosage of CBD treating is 5 μM-10 μM.

7. A method of screening and identifying small molecule having antiviral properties, the method comprising:
Step (a): Isolating a plurality of mouse embryo fibroblasts (MEFs) from mice, and choosing MEFs at passage 3 for cell cultures and chemical treatment;
Step (b): Cell culturing and chemical treating said isolated MEFs to culture a plurality of cells;
Step (c): Determining the cell's proliferation rate and an optimal dosage of small molecules by counting the cells and calculating a population doubling level of said cells of step (b);
Step (d) Extracting RNA from the cells of step (b) to obtain a plurality of RNA samples, followed by DNase digesting to remove DNA contaminant in the RNA samples, followed by reverse transcription to obtain a plurality of transcribed cDNA, and carrying out the quantitative polymerase chain reaction (qPCR) test for the transcribed cDNA;
Step (e) RNA sequencing for the RNA samples of step (e) to examine the quality and sequence of RNA;
Step (f) treating mice with solution containing small molecule having antivirus properties;
Step (g): dissecting said mice for preparation of tissues extraction of RNA;
Step (h): Examining and detecting inhibitory effect of said small molecule on viral infection.

8. The method according to claim 7 wherein said smaller molecule is Cannabidiol (CBD).

9. The method according to claim 8 wherein said viral infection is COVID-19 caused by nCoV virus.

10. The method according to claim 9 where optimal dosage of said small molecule in the step (f) is 5 μM-10 μM.
